(19) United States
(12) Patent Application Publication
(10) Pub. No.: US 2020/0342977 A1
(43) Pub. Date: Oct. 29, 2020

(54) SYSTEM, COMPUTER-READABLE STORAGE MEDIUM, AND METHOD

(71) Applicant: SoftBank Group Corp., Tokyo (JP)

(72) Inventor: Masayoshi Son, Tokyo (JP)

(21) Appl. No.: 16/596,771

(22) Filed: Oct. 9, 2019

(30) Foreign Application Priority Data
Apr. 26, 2019 (JP) 2019-085687

(51) Int. Cl. G16H 20/60; G09B 19/00; G06Q 50/12; G06Q 30/02

(52) U.S. Cl. CPC G16H 20/60; G06Q 30/02; G06Q 50/12; G09B 19/0092

(57) ABSTRACT

A system includes an information acquiring unit that acquires information indicating a past meal history of a user, a determination unit that determines a recommend meal menu for the user from a plurality of pre-registered meal menus based on information acquired by the information acquiring unit, and a provision unit that provides information indicating a meal menu determined by the determination unit. A method performed by a computer comprises acquiring information indicating a past meal history of a user. The method comprises determining the recommend meal menu for the user from the plurality of pre-registered meal menus based on the acquired information. The method comprises providing information indicating the determined meal menu.

The contents of the following Japanese patent application are incorporated herein by reference: Patent Application No. 2019-085687 filed on Apr. 26, 2019.

### BACKGROUND

### 1. Technical Field

The present invention relates to a system, a computer-readable storage medium, and a method.

### 2. Related Art

An image capture system is known that allows food analysis and volume measurement by capturing an image of a table (for example, see Patent Literature 1).
Patent Literature 1: Japanese Patent Application Publication No. 2018-190259

### SUMMARY

It is desired to provide recommend meal menus suitable for individual users.

### BRIEF DESCRIPTION OF THE DRAWINGS

FIG. 1 schematically illustrates one example of the system 10.

FIG. 2 schematically illustrates one example of a functional configuration of a server 40.

FIG. 3 schematically illustrates one example of a flow of processes by the server 40.

FIG. 4 illustrates a data structure of meal history information stored in a database 50.

FIG. 5 illustrates a data structure of exercise history information stored in the database 50.

FIG. 6 illustrates a data structure of user information stored in the database 50.

FIG. 7 illustrates a data structure of food information stored in the database 50.

FIG. 8 schematically illustrates one example of a flow of processes by the server 40.

FIG. 9 schematically illustrates a display content of a recommended meal menu displayed on a user terminal 82.

FIG. 10 schematically illustrates a recommended meal menu content displayed on the user terminal 82.

FIG. 11 schematically illustrates one example of a hardware configuration of a computer 1200 to function as a server 40.

### DESCRIPTION OF EXEMPLARY EMBODIMENTS

Hereinafter, the present invention will be described with reference to embodiments of the invention. However, the following embodiments should not to be construed as limiting the claimed invention. Also, all the combinations of the features described in the embodiments are not necessarily essential for means provided by aspects of the invention.

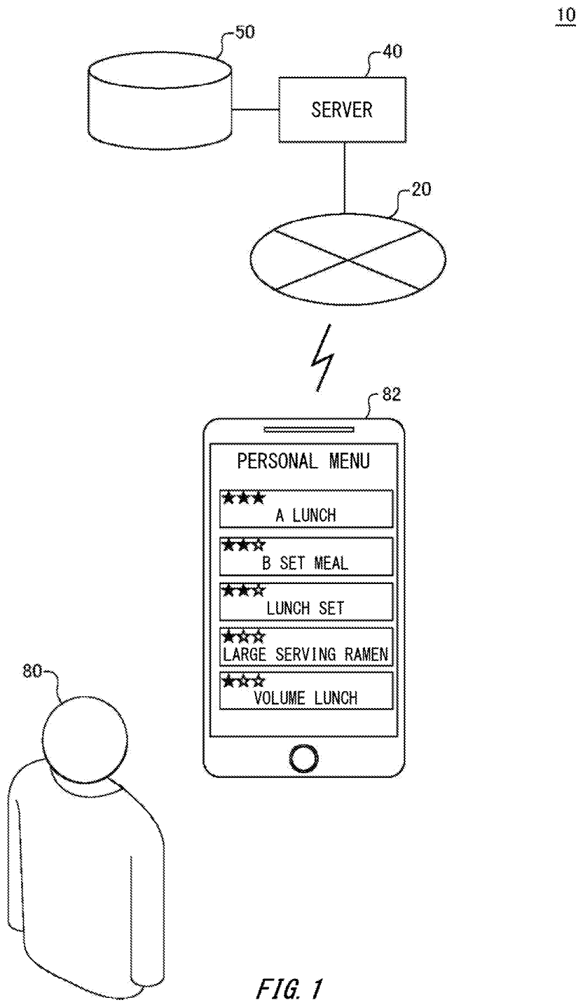

FIG. 1 schematically illustrates one example of a system 10. The system 10 of the present embodiment includes a server 40, a database 50, and a user terminal 82. The system 10 provides information indicating a meal menu suitable for each user 80. Specifically, the system 10 provides information indicating a meal menu recommended to the user 80 based on a meal history of the user 80.

The server 40 and the user terminal 82 communicate with each other via a network 20. The network 20 may include a mobile communication network and the Internet. The mobile communication network may conform to any of a 3G (3rd Generation) communication system, an LTE (Long Term Evolution) communication system, a 5G (5th Generation) communication system, a 6G (6th Generation) communication system and subsequent communication systems. The user terminal 82 may access the mobile communication network via a wireless base station or via a WiFi (registered trademark) connection.

The user terminal 82 is information equipment used by the user 80. The user terminal 82 may be a mobile communication device such as a mobile phone such as a smartphone and a tablet terminal. The user terminal 82 may be a portable communication device such as a laptop personal computer. The user terminal 82 may be stationary communication equipment such as a personal computer.

When having the meal, the user 80 captures an image of a served food by using the user terminal 82. The user terminal 82 transmits, to the server 40, meal menu image information including captured image data. Upon receipt of the meal menu image information from the user terminal 82, the server 40 identifies a food consumed by the user 80 and identifies a nutrients included in the identified food by analyzing the image data included in the meal menu image information. The server 40 stores, in the database 50, the meal history information including information indicating the identified food, information indicating a nutrient, date and time information, positional information indicating the user terminal 82 in association with identification information of the user 80.

When the user 80 is about to have a meal, the user 80 requests the server 40 for a meal menu recommended to the user 80 by operating the user terminal 82. The user 80 launches application software installed in the user terminal 82 and performs an operation for acquiring the meal menu recommended to the user 80. In the present embodiment, the meal menu recommended to the user 80 may be referred to as a “recommended meal menu.”

When the user 80 performs an operation for acquiring the recommended meal menu for the user 80, the user terminal 82 transmits, to the server 40, information for requesting the recommended meal menu for the user 80 according to a control of the application software. Upon receipt of the request information, the server 40 determines the recommended meal menu for the user 80 based on the meal history information of the user 80. For example, the server 40 refers to the database 50 and determines the recommended meal menu for the user 80 based on the meal history information associated with the identification information of the user 80 included in the request information. The server 40 transmits, to the user terminal 82, information indicating the determined recommended meal menu.

The system 10 can provide information indicating the meal menu suitable for each user 80 based on the meal history of the user 80.

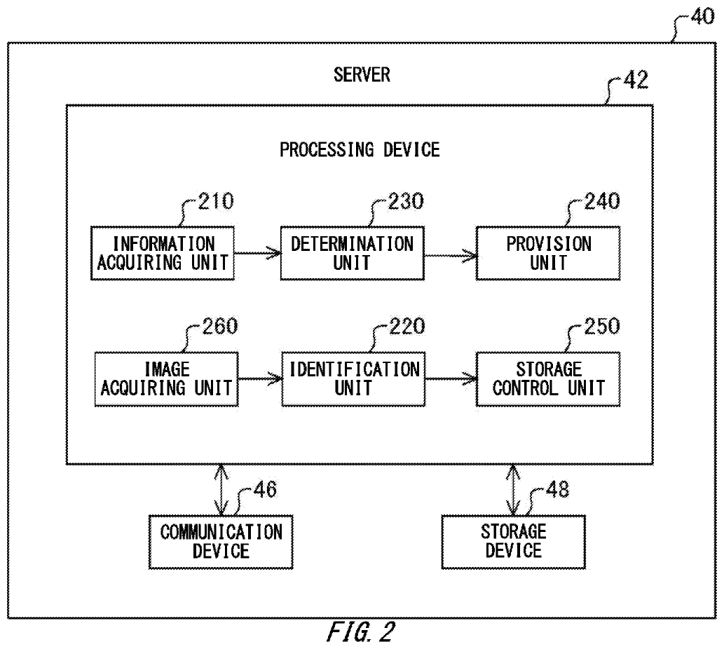

FIG. 2 schematically illustrates one example of a functional configuration of a server 40. The server 40 includes a processing device 42, a communication device 46, and a storage device 48.

The processing unit 42 may be realized by an arithmetic processing device such as a processor. The storage device 48 includes non-volatile storage media and/or volatile storage media. The processing device 42 performs processes using information stored on the storage device 48. The information stored in the storage device 48 includes an operating system (OS), communication control software, application software operating on the OS, and the like. The communication device 46 is responsible for communication with the user terminal 82. The communication device 46 supplies information received from the user terminal 82 to the processing device 42 through the network 20. The communication device 46 transmits through the network 20, to the user terminal 82, information addressed to the user terminal 82 generated by the processing device 42.

The processing device 42 includes an information acquiring unit 210, a determination unit 230, a provision unit 240, and a storage control unit 250. The information acquiring unit 210 acquires information indicating a past meal history of the user 80. The determination unit 230 determines the meal menu to be recommended to the user 80 from a plurality of pre-registered meal menus based on the information acquired by the information acquiring unit 210. The provision unit 240 provides information indicating the meal menu determined by the determination unit 230.

The information acquiring unit 210 further acquires information indicating a goal of the user 80. The determination unit 230 determines the meal menu that is suited to the goal of the user 80 from the plurality of meal menus based further on information indicating the goal of the user 80. The goal of the user 80 is, for example, a goal such as a strengthening muscle, a losing weight or the like. The goal of the user 80 is, for example, a goal that the user 80 wishes to achieve by adjusting at least meal contents.

The image acquiring unit 260 acquires information indicating an image of the food consumed by the user 80. The identification unit 220 identifies the nutrient ingested from the food by recognizing the food consumed by the user 80 in the image. The storage control unit 250 stores information obtained by associating nutritional information indicating the nutrient taken by the user 80, the identification information of the user 80, information indicating date and time when the user 80 has the food in the database 50 as information indicating the meal history. The database 50 is one example of a “storage unit.” The nutritional information may include information indicating a type of a nutrient and information indicating the amount of each nutrient.

The information acquiring unit 210 acquires, as the information indicating the meal history, the nutritional information and the date and time information that are stored in the database 50 in association with the identification information of the user 80. When received the request information from the user 80, the determination unit 230 determines meal menu recommended to a next meal of the user 80 from a plurality of meal menus based on the nutritional information and the date and time information associated with the identification information of the user 80.

When the user 80 is in a store where the meal is served, the provision unit 240 determines the meal menu to be recommended to the user 80 from the plurality of menus that the store is ready to serve. When the user 80 is not in the store serving the meal, the provision unit 240 determines the meal menu to be recommended to the user 80 from the plurality of menus available in a plurality of stores associated with a current position of the user 80. Accordingly, if the user 80 is in a restaurant, it is possible to provide a meal menu optimized for each user 80 from meal menus available in the restaurant.

The determination unit 230 determines a first meal menu recommended when the user 80 has a meal at his living location and a second meal menu recommended when the user 80 has the meal outside his living location. The living location is, for example, a home of the user 80. The home of the user 80 is pre-registered using the user terminal 82. The provision unit 240 provides the first meal menu and the second meal menu to the user 80 according to the order of priority based on the current position of the user 80. For example, the provision unit 240 provides the first meal menu and the second meal menu to the user 80 according to the order of priority based on a distance between the current position and the living location. Specifically, the farther the current position is from the living location, the higher the priority given to provision of the second meal menu is over the priority given to provision of the first meal menu.

The provision unit 240 provides the first meal menu and the second meal menu to the user 80 according to the order of priority based on the current position of the user 80 and a current time. For example, the provision unit 240 provides the first meal menu and the second meal menu to the user 80 according to the order of priority based on a distance between the current position and the living location, and a current time. For example, the meal history includes information indicating a time when the user 80 had the past meal. The provision unit 240 provides the second meal menu prior to the first meal menu to the user 80 according to the order of priority based on a time required for moving from the current position to the living location, the current time, and the time when the user 80 had the past meal.

For example, it is assumed that the user 80 requests the recommended meal menu using the user terminal 82 when leaving the office. Upon reception by the server 40 of the request information from the user terminal 82, the determination unit 230 identifies the time required for moving from the current position to the home and an expected time at which the user 80 arrives at the home. The provision unit 240 provides the first meal menu prior to the second meal menu sets the priority given to provision of the first meal menu higher than the priority given to provision of the second meal menu if time between an average value of the meal time information included in the meal history information and the expected time at which the user 80 arrives at the home is longer than predetermined time.

The first meal menu may include a third meal menu including a food obtained by cooking a food material and a fourth meal menu using a premade food. The provision unit 240 provides the third meal menu and the fourth meal menu to the user 80 according to the order of priority based on the distance between the current position and the living location of the user 80 and the current time. For example, if the user 80 requests the recommended meal menu using the user terminal 82 when leaving the office, the determination unit 230 identifies the time required for moving from the current position to the home and the expected time at which the user 80 arrives at the home. The determination unit 230 determines cooking time required for preparing the recommended meal menu for the user 80. The provision unit 240 sets the priority given to provision of the third meal menu higher than the priority given to provision of the fourth meal menu according to the order of priority based on the average value of the meal time information included in the meal history information, the cooking time, and the expected time at which the user 80 arrives at the home. For example, the provision unit 240 sets the priority given to provision of the third meal menu higher than the priority given to provision of the fourth meal menu when a difference between the average value of the meal time information and a time which is the cooking time after the expected time at which the user 80 arrives at the home is longer than the predetermined time.

The determination unit 230 determines the meal menu to be recommended to the user 80 from the plurality of meal menus based on the information acquired by the information acquiring unit 210 and information indicating what the user 80 plans to eat. The provision unit 240 provides information indicating the meal menu determined by the determination unit 230 in association with information indicating behavioral plan.

The information indicating behavioral plan may include information indicating a meal menu to be taken by the user 80 after having a next meal. For example, when the server 40 receives the request information of the recommended meal menu at lunch time, the determination unit 230 determines a recommended meal menu for dinner and a recommended meal menu for lunch. The provision unit 240 transmits, to the user terminal 82, information indicating the recommended meal menu for dinner and the recommended meal menu for lunch. Accordingly, the server 40 can recommend the user 80 to have a meal of the recommended meal menu for lunch at lunch time on the assumption that the user 80 has a meal of the recommended meal menu for particular dinner at dinner time. For example, when the user 80's goal is to lose weight, the server 40 can recommend a meal of a high calorie meal menu at lunch time based on an assumption that the user 80 has a meal of a low calorie meal menu at dinner time.

The information indicating behavioral plan may include information indicating the amount of exercise performed by the user 80 after having the next meal. The information indicating the amount of exercise may include information indicating a walking distance of the user 80. The walking distance may be a distance between the closest station to the user 80's home and the user's home. The user 80's home and closest station may be pre-registered in the user terminal 82 and/or the server 40. The information indicating the amount of exercise may include training information such as a running distance and a running speed at a training gym. Information indicating a training menu performed by the user 80 may be pre-registered in the user terminal 82 and/or the server 40.

The information acquiring unit 210 further acquires information indicating the past exercise history of the user 80. The determination unit 230 determines the meal menu to be recommended to the user 80 from the plurality of meal menus based on information indicating the past exercise history of the user 80. For example, the determination unit 230 may determine the amount of exercise of the user 80 on that day based on the exercise history information and may determine a meal menu to be recommended to the user 80 such that the larger the identified amount of exercise is, the higher the calories of the recommended meal menu is.

The information acquiring unit 210 further acquires information indicating past meal histories of one or more other users 80 associated with the user 80. The determination unit 230 determines the meal menu to be recommended to the user 80 from the plurality of meal menus based on information indicating a past meal history of other users 80. For example, based on information indicating meal histories of the user 80 and the user 80's family member the determination unit 230 determines one meal menu as the meal menu to be recommended to the user 80 when the user 80 has the meal at the living location. On the other hand, as meal menus to be recommended to the user 80 and his/her family members when they are having a meal outside their living location, the determination unit 230 determines meal menus available in a single store among meal menus recommended to the user 80 and his/her family members determined based on information on their meal histories. The provision unit 240 provides, to the user 80, the meal menus available in a single store prior to other meal menus. Accordingly, the server 40 can provide, to the user 80, the meal menus suitable for the user 80 and the user 80's family member.

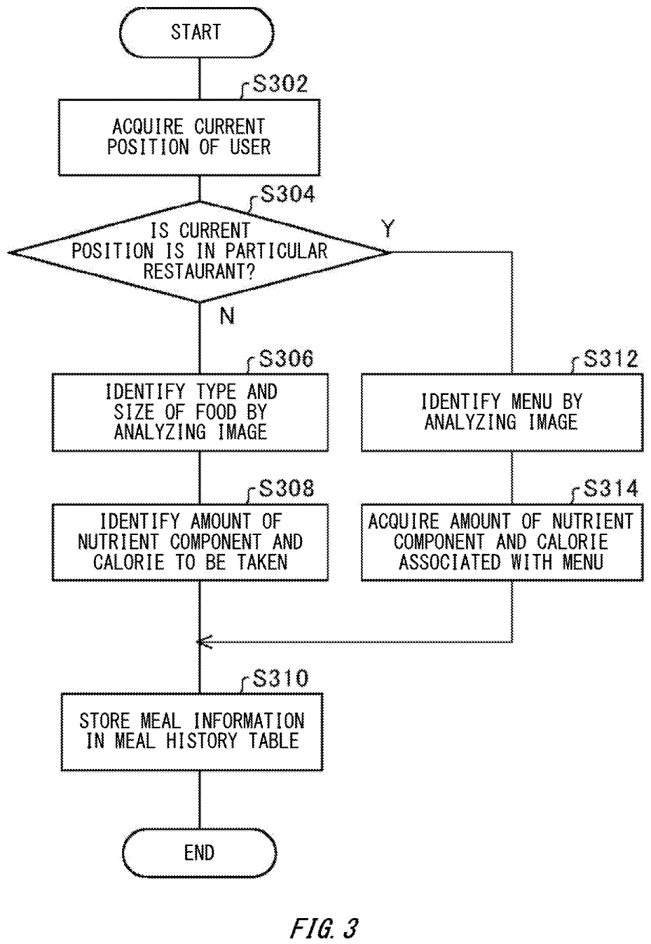

FIG. 3 schematically illustrates one example of a flow of processes performed of a server 40. FIG. 3 schematically illustrates a flow of processes performed when the server 40 acquired an image of a meal from the user terminal 82. Each process illustrated in FIG. 3 is performed under the control of the server 40.

In step (step may be abbreviated to S) 302, an identification unit 220 acquires a current position of a user 80. For example, meal menu image information transmitted by a user terminal 82 includes current position information of the user terminal 82 whose current position is measured by the user terminal 82. The identification unit 220 identifies the current position of the user 80 based on the current position information included in the meal menu image information.

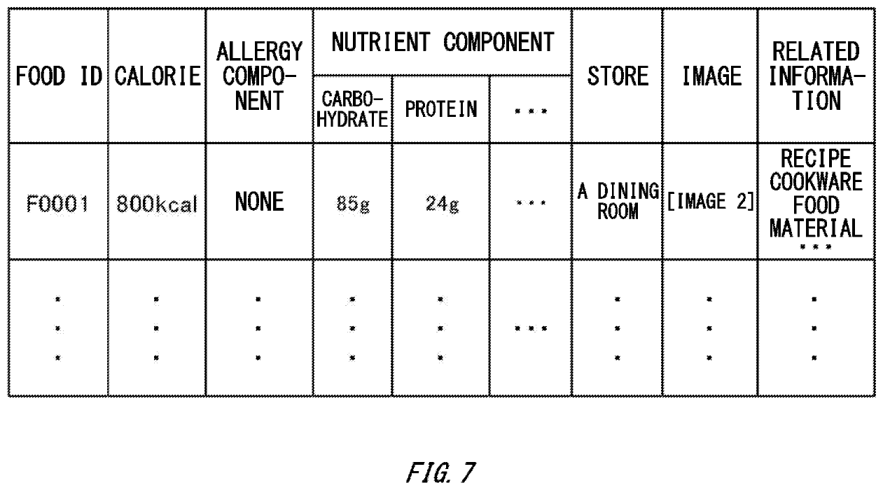

In S304, the identification unit 220 determines whether the current position of the user 80 is in a particular restaurant. The particular restaurant is a restaurant that can serve menus indicated by information registered in a database 50. The menu information available in the particular restaurant is described with reference to a table in FIG. 7 and the like.

When the current position of the user 80 is not in a particular restaurant in S306, the identification unit 220 recognizes a food and identifies a type and a size of the food by analyzing image data included in the meal menu image information. For example, the identification unit 220 identifies the type of the food based on color information of a subject in the image data included in the meal menu image information. The identification unit 220 identifies the size of the food based on subject distance information included in the meal menu image information, imaging view angle information, and the size of the food in the image data. The size of the food may be information indicating a volume of the food. The identification unit 220 may identify the size of the food by taking into account a size of an object other than the food shown in the image. The identification unit 220 may identify the size of the food by taking into account sizes of images of hands or fingers of the user 80 shown in the image. The identification unit 220 may identify the size of the food by taking into account a size of a plate image shown in the image. In this manner, the identification unit 220 may identify the size of the food by taking into account the size of the object when the image includes the image of the object with a known size.

The size of the food may be discrete information corresponding to the volume of the food instead of information indicating the volume itself. For example, when a food is boiled egg, “S size” and the like can exemplify a food size and when a food is rice, “large serving” and the like can exemplify a food size.

In S308, the identification unit 220 identifies the amount of nutrient components and the calorie ingested from the food based on the type and size of the food identified in S306. For example, food information indicating the amount of nutrient components and calorie ingested from the food per unit volume may be pre-registered in the database 50 for each food. The identification unit 220 identifies the amount of nutrient components and the calorie ingested from the food based on the food information registered in the database 50 and the size of the food identified in the image data.

In S310, a storage control unit 250 causes the database 50 to store the type and size of the food identified in S306, the amount of nutrient components and the calorie identified in S308, and current time information.

When it is determined in S304 that the current position is in the particular restaurant, in S312, the identification unit 220 identifies a menu served to the user 80 from menus available in the restaurant by analyzing the image data included in the meal menu image information. The identification unit 220 identifies a menu provided to the user 80 by matching the image data stored in the database 50 with the image data included in the meal menu image information received from the user terminal 82 when, for example, the database 50 stores the image data of the menu provided in the restaurant in association with the information indicating the amount of nutrient components and the calorie ingested from the food in the menu. In S314, the identification unit 220 acquires, from the database 50, the amount of nutrient components and the calorie associated with the menu identified in S312. After performing the S314, the process proceeds to S310.

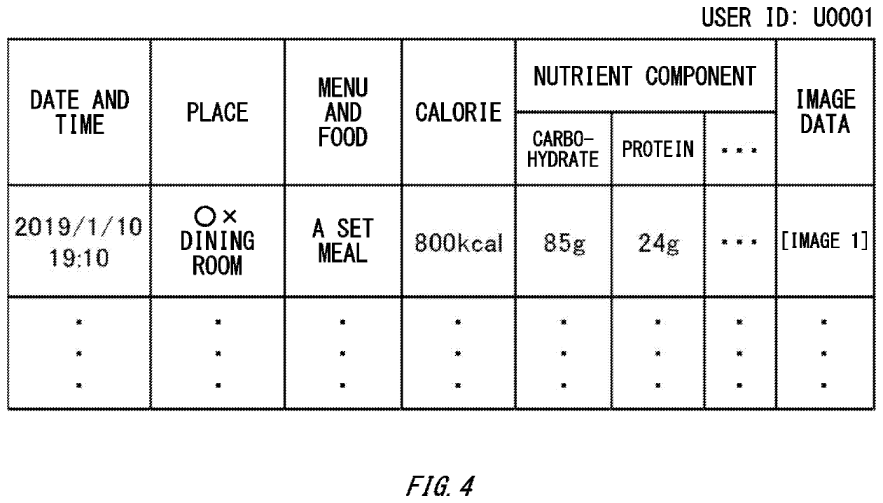

FIG. 4 illustrates a data structure of meal history information stored in a database 50. The meal history information includes date and time information, place information, menu and food information, calorie information, nutrient component information and image data in association with one another. FIG. 4 illustrates meal history information associated with “U0001” that is identification information of a specific user 80. The database 50 stores the meal history information illustrated in FIG. 4 in association with the identification information of each of other users.

The date and time information may be information indicating date and time when the server 40 receives the meal menu image information. The place information may be information determined based on the current position information of the user terminal 82 included in the meal menu image information. The place information may include information indicating the latitude and longitude of the current position of the user terminal 82. The place information may include the name of a restaurant determined from the current position information of the user terminal 82. The place information may include information indicating names of pre-registered locations such as a “home” or an “office.”

The menu and food information may include information identifying a meal menu taken by the user 80. For example, the menu and food information may include a name of a menu provided in a restaurant. The menu and food information may include a name of a food included in the menu provided in the restaurant. The menu and food information may include a name of a menu identified by the identification unit 220 based on the food identified from the meal menu image information. For example, the menu and food information may include a name such as “ginger pork.”

The calorie information indicates the calorie identified by the identification unit 220. The nutrient component information may include information indicating a mass of each type of the plurality of nutrients ingested. Examples of types of nutrients include carbohydrates, proteins, lipids, dietary fibers, various vitamins, various minerals and the like.

As has been described with reference to FIG. 3 and the like, the identification unit 220 may generate the meal history information based on the meal menu image information. As image data of a meal menu image, image data posted to a social network service (SNS) may be used. The server 40 may generate the meal history information by acquiring, from an SNS server, posted image data, posting date and time, and information indicating position of posting.

The user 80 may manually input meal contents to the user terminal 82. For example, the user 80 inputs, to the user terminal 82, the food information such as “ginger pork,” “rice,” or “miso soup” and information indicating the amount of the food ingested (for example, “large serving” “medium serving” “small serving” and the like) as the meal contents. The user terminal 82 receives meal information by the user 80 through a manual input and transmits the thus obtained information to the server 40. The server 40 may generate the meal history information based on the meal information received from the user terminal 82.

The identification unit 220 may generate the meal history information based on purchase history information. For example, the server 40 acquires, from an external server, information indicating products purchased by the user 80 at stores such as a supermarket and a convenience store. For example, the server 40 receives information indicating a type of a boxed lunch purchased by the user 80 at the convenience store and a nutritional component and a calorie ingested from the boxed lunch. The identification unit 220 generates the meal history information based on the information received from the external server.

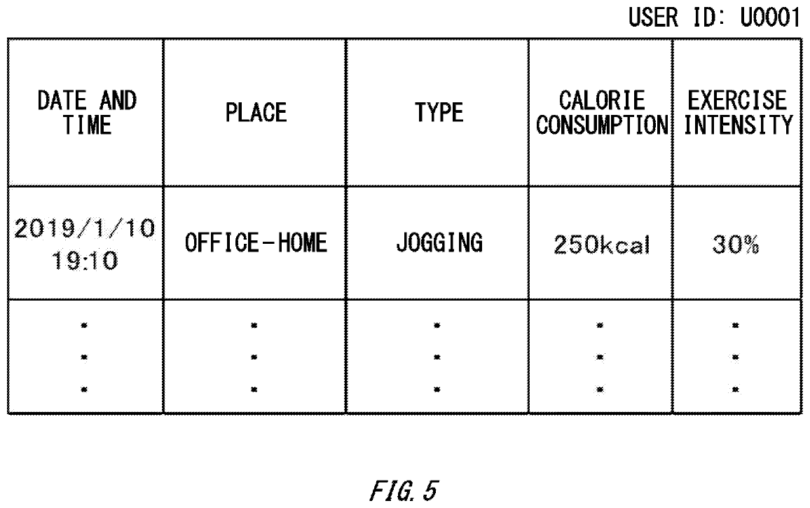

FIG. 5 illustrates a data structure of exercise history information stored in a database 50. The exercise history information includes date and time information, place information, type information, calorie consumption information, and exercise intensity information in association with one another. FIG. 5 illustrates exercise history information associated with “U0001” that is identification information of a specific user 80. The database 50 stores the exercise history information illustrated in FIG. 5 in association with identification information of each of other users.

Upon reception of exercise information of the user 80 from the user terminal 82, the server 40 generates the exercise history information illustrated in FIG. 5 and stores the information in the database 50. For example, as the exercise information, the server 40 may periodically receive, from the user terminal 82, information managed by an exercise management application installed on the user terminal 82.

The date and time information may be information indicating date and time or a period of exercises of the user 80. The place information may be information indicating a place where the user 80 exercises. The place information may be determined from the positional information of the user terminal 82. The place information may include information indicating the latitude and the longitude of a location where the user terminal 82 is present. The place information may include a name of an exercise facility determined from the positional information of the user terminal 82. The place information may include information indicating names of pre-registered locations such as “home” and “OX training gym.”

The type information may include information indicating a type of the exercise content of the user 80. For example, the type information may include a name of a jogging menu. The type information may include information indicating a training menu performed by the user in an exercise facility. For example, the type information may include information indicating a training menu such as “400 meters interval running×5.”

The calorie consumption information includes information indicating a calorie consumed by the user performing an exercise. The exercise intensity information includes information indicating intensity of an exercise performed by the user 80. The exercise intensity information may be measured based on a heart rate or the like. The server 40 may acquire the calorie information and the exercise intensity information from the exercise management application.

The server 40 may generate the exercise history information using information posted to a social network service (SNS). For example, the server 40 may generate the exercise history information by acquiring, from an SNS server, posted character information, image data, posting date and time, and positional information at the time of posting. For example, the server 40 may generate the exercise history information based on the post content “Today I jogged from my office to home.”

The user 80 may manually input the exercise information to the user terminal 82. For example, as the exercise information, the user 80 inputs, to the user terminal 82, type information such as “jogging” and information indicating the amount of exercise such as a running distance and the number of steps. The user terminal 82 receives the exercise information from the user 80 through a manual input and then transmits the received information to the server 40. The server 40 may generate the exercise history information based on the exercise information received from the user terminal 82.

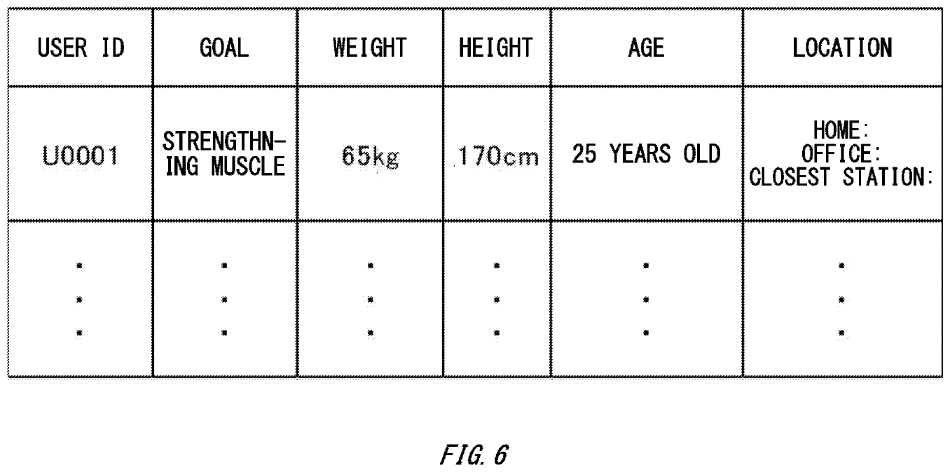

FIG. 6 illustrates a data structure of user information stored in a database 50. The user information includes a user ID, target information, weight information, height information, age information, and location information in association with one another.

The user ID is the identification information of the user 80. The target information indicates a goal of the user 80. The “goal” in this case is a goal relating to a meal. The degree of achievement of the “goal” is the goal influenced by the meal content. The weight information indicates a weight of the user 80. Examples of target information include “strengthening muscle,” “losing weight,” “diabetes control,” “adult disease prevention,” “de-allergy” “emphasis on taste” and the like. The height information indicates a height of the user 80. The age information indicates an age of the user 80.

The location information includes information indicating identification information, types, names, and locations of a home, an office, a closest station, an exercise facility to be used and the like. The home is one example of a living location of the user 80.

The determination unit 230 determines a recommended meal menu to each user 80 by considering the user information. For example, when a goal of the user 80 is the “strengthening muscle,” the determination unit 230 identifies the amount of protein to be taken from a next meal based on a weight, a height, an age and the exercise history information and determines a meal menu allowing the user to take the identified amount of protein. When a goal of the user 80 is the “emphasis of taste,” the determination unit 230 judges a taste of the user 80 based on the meal history information and determines a meal menu that meets the taste of the user 80. For example, the determination unit 230 judges food materials that the user 80 likes and dislikes and determines a meal menu including the food material that the user 80 likes but not including the food material that the user 80 dislikes.

The identification unit 220 may identify a distance between, for example, a home and a closest station or between the home and an office based on positional information of a location registered in the location information, identify a calorie consumption and exercise intensity based on the identified distance, and then generate exercise history information including the identified calorie consumption and exercise intensity. The identification unit 220 may identify calorie consumption and exercise intensity based on information indicating a training menu provided in an exercise facility registered in the location information and generates exercise history information including the identified calorie consumption and exercise intensity.

FIG. 7 illustrates a data structure of food information stored in a database 50. The food information is obtained by associating a food ID, calorie information, allergy component information, a nutritional component, store information, image information, and related information from one another.

The food ID is identification information of a food. The “food” described herein includes information indicating a food material, a boxed lunch, a side dish, and a beverage sold at a store such as a convenience store and a supermarket and a newly created menu assuming cooking by the user 80 in addition to a meal menu provided in a store such as a restaurant. The menu assuming cooking by the user 80 includes a registered menu of a meal prepared by the user 80's cooking, a meal menu registered in a cooking community site and the like.

The calorie information relates to a calorie that can be taken from the meal menu. The allergy component information includes information indicating a type of an allergy component of a food included in a meal menu. The nutrient component information includes information indicating a mass of each type of a plurality of nutrients ingested. As described above, examples of nutrient types include carbohydrates, proteins, lipids, dietary fibers, various vitamins, various minerals and the like.

The store information identifies a store. A store is a restaurant, a convenience store, a supermarket or the like. The store information may include a store name and positional information. The image information is an image of a corresponding product. The image information may be, for example, an image of a meal menu provided in a restaurant.

The related information is information relevant to a corresponding food. Examples of related information include, for example, recipe information of the corresponding food, information indicating a cookware for cooking of the corresponding food, information indicating a food material to prepare the corresponding food, information indicating seasonings used to season the corresponding food, information indicating a food material frequently purchased by a person who favors the corresponding food, and information indicating a cookware frequently used by a person who favors the corresponding food.

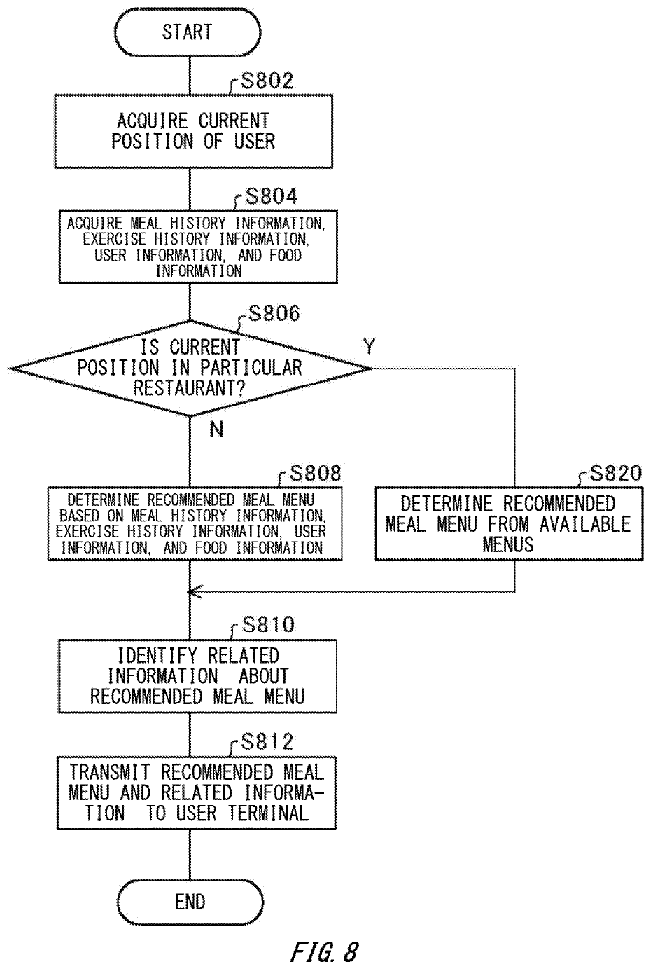

FIG. 8 schematically illustrates one example of a flow of processes of a server 40. FIG. 8 schematically illustrates a flow of processes performed when a server 40 receives, from a user terminal 82, request information indicating a recommended meal menu. Each process illustrated in FIG. 8 is mainly performed by the server 40.

In S802, a determination unit 230 acquires a current position of a user 80. For example, request information of a recommended meal menu transmitted from the user terminal 82 includes current position information of the user terminal 82 whose current position is measured by the user terminal 82. The determination unit 230 identifies a current position of the user 80 based on current position information included in the request information.

In S804, an information acquiring unit 210 acquires, from a database 50, meal history information, exercise history information, user information, and food information of the user 80. For example, the information acquiring unit 210 acquires meal history information, exercise history information, user information, and also food information that are stored in the database 50 in association with identification information of the user 80.

In S806, the determination unit 230 determines whether a current position of the user 80 is in a particular restaurant. The particular restaurant is a restaurant that can serve menus indicated by information registered in a database 50. For example, the determination unit 230 determines whether the current position of the user 80 is at a restaurant that can serve menus which are included in menu information registered in the food information illustrated in FIG. 7.

When the current position of the user 80 is not in a particular restaurant, in S808, the determination unit 230 determines a recommended meal menu for the user 80 based on a current time, the current position of the user 80, meal history information, exercise history information, user information, and food information. As the recommended meal menu, the determination unit 230 determines, for example, a meal menu that is suited to a goal of the user information. As described above, when the goal of the user 80 is a strengthening muscle for example, a meal menu having the amount of protein to be ingested that is equal to or above a predetermined amount is determined as the recommended meal menu. At this time, as the predetermined amount for defining the lower limit amount of the protein, the determination unit 230 may set an amount according to exercise intensity information in exercise history information.

When the goal of the user 80 is de-allergy, the determination unit 230 determines a meal menu not including an allergic material and/or a meal menu made of a food not including an allergic material. When determining a meal menu for eating at home, the determination unit 230 may identify a store located within a predetermined distance from the user 80's home, identify a premade food or a food material that can be purchased at the store by referring to the food information, set a recommended meal menu including the identified food and/or a meal menu obtained by cooking the identified food material as a recommended meal menu. Based on stock information of a store located within a predetermined distance from the user 80's home, the determination unit 230 may identify a food or a food material predicted to be in stock at the store when the user 80 arrives at the store by taking into account a time required for the user 80 to move from a current position to the store, and determine a recommended meal menu including the identified food and/or a meal menu obtained by cooking the identified food material as a recommended meal menu.

At this time, the determination unit 230 identifies a timing at which the user 80 has a next meal based on a current time. The determination unit 230, for example, identifies whether the next meal of the user 80 is a breakfast, a lunch, or a dinner based on the current time and identifies an average time the user 80 has a next meal based on date and time information included in meal history information. The determination unit 230, for example, identifies an average time of times at which the user 80 had meals during a specified time frame for having a meal among a breakfast time frame, a lunch time frame, and a dinner time frame (sometimes referred to as “normal meal time”) based on the meal history information.

The determination unit 230 determines a priority of a recommend meal menu based on a time required for moving from a current position to a home and a pre-registered time required for cooking a food material. For example, respective priorities are determined for a recommended meal menu that requires cooking of a food material, a recommended meal menu including a boxed lunch and a side dish that are already cooked and bought at a store near the user's home, and a recommended meal menu available at a restaurant or the like outside the home.

For example, if a time which is the length of time required for moving to the home after the current time is at or before a time point which is a predetermined length of time before the normal meal time, the determination unit 230 sets the higher priority to a recommended meal menu when the user has a meal at home than a priority of a recommended meal menu when the user has a meal outside a home.

If a time which is the length of time required for moving to the home and required for a cooking time after the current time is after the normal meal time and a time which is the length of time required for moving to the home after the current time is before the normal meal time, the determination unit 230 sets the priority given to a recommended meal menu including a cooked food higher than the priority given to a recommended meal menu that requires cooking of a food material.

If a time which is the length of time required for moving to the home and required for a cooking time after the current time is after the normal meal time, the determination unit 230 sets a priority given to a recommended meal menu when the user has a meal outside a home higher than a priority given to a recommended meal menu when a user has the meal at home. The determination unit 230 sets a higher priority of a meal menu that can be provided by a restaurant present within a range where the user can go until the normal meal time than a priority of a meal menu that can be provided by a restaurant present outside a range where the user can go until the normal meal time.

The determination unit 230 identifies a recommended meal menu for the user 80's family member. The determination unit 230 sets a priority of the same recommended meal menu among a recommended meal menu for the user 80 and a recommended meal menu for the user 80's family member higher than priorities of other recommended meal menus with respect to a recommended meal menu when a user has a meal at home. With respect to a recommended meal menu when the user has a meal outside a home, the determination unit 230 sets a priority of a recommended meal menu available at the same restaurant among the recommended meal menu for the user 80 and the recommended meal menu for the user 80's family member higher than priorities of other recommended meal menus. The determination unit 230 may determine a priority of a recommended meal menu based further on a current position of the user 80's family member.

Subsequently, in S810, the determination unit 230 identifies related information associated with the recommended meal menu determined in S808. As described above, examples of the related information can include recipe information, food material information, seasoning information, cookware information, store information and the like.

In S812, the provision unit 240 causes the server to transmit the recommended meal menu determined by the determination unit 230 and the related information identified in S810 to the user terminal 82 through the communication device 46.

When in S806, it is determined that the current position of the user 80 is in a particular restaurant, in S820, the determination unit 230 determines the recommended meal menu for the user 80 from meal menus that can be provided by the restaurant based on the current time, the current position of the user 80, the meal history information, the exercise history information, and the user information. The determination unit 230 determines, as the recommended meal menu, for example, a meal menu suitable for a goal included in the user information from the meal menus that can be provided by the restaurant. After determining the recommended meal menu, the process proceeds to S810.

The determination unit 230 may determine a next meal menu by taking into account a recommended meal menu taken after the next meal when the recommended meal menu is determined in S808 and/or S820. For example, when a next meal is a lunch, the determination unit 230 may determine the recommended meal menu of lunch by taking into account the recommended meal menu of dinner. In this case, the provision unit 240 transmits, to the user terminal 82, the recommended meal menu for dinner and the recommended meal menu for lunch. Accordingly, when taking the recommended meal menu for lunch, the user 80 can know what meal menu to take for dinner. For example, if the user has a meal of a high calorie meal menu for lunch, the user can know an approximate amount of meal for dinner. Therefore, it is possible to easily determine whether to have a meal of a high calorie meal menu for lunch.

The determination unit 230 may determine a meal menu of a next meal by taking into account information indicating the amount of exercise performed after a next meal when determining the recommended meal menu in S808 and/or S820. The determination unit 230 determines the recommended meal menu, for example, by taking into account the amount of exercise performed after the next meal. The determination unit 230 identifies an exercise that can be performed by the user 80, for example, based on the exercise history information of the user 80. The determination unit 230 identifies a calorie expected to be consumed by the user 80 performing an identified exercise. The determination unit 230 may determine an upper limit of a range of a calorie that can be taken at a next meal based on the identified calorie and then determine a meal menu having a calorie to be ingested within the range of the expected calorie as the recommended meal menu.

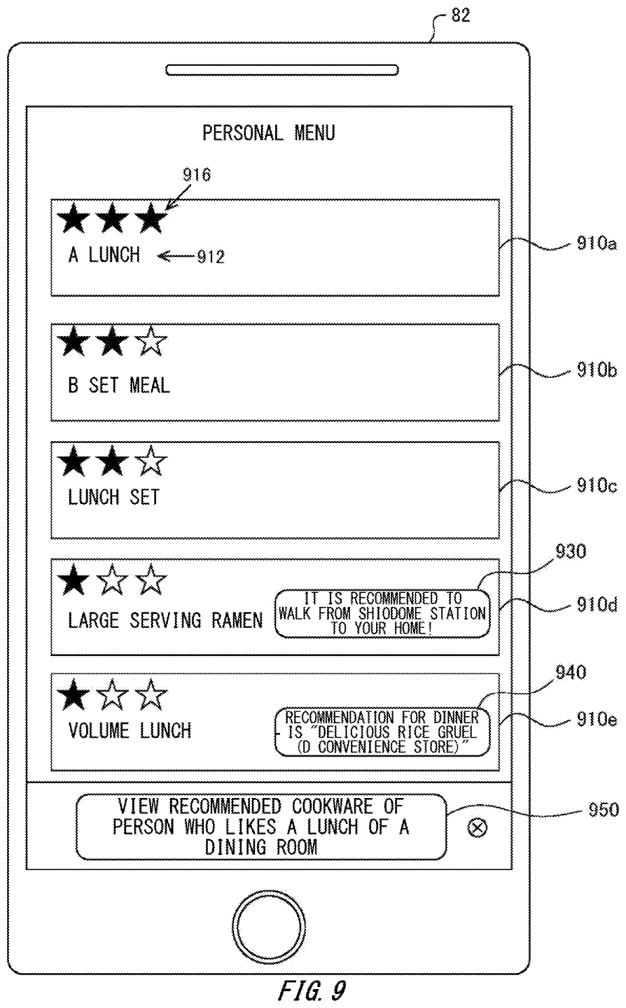

FIG. 9 schematically illustrates display contents of a recommended meal menu displayed on a user terminal 82. FIG. 9 illustrates one example of a display screen of recommended meal menus for lunch when a current position of the user 80 is in a particular restaurant.

The user terminal 82 displays objects 910a to 910e indicating recommended meal menus based on information indicating the recommended meal menus received from the server 40 according to control of application software. The user terminal 82 displays each object 910 in an order of priority. The user terminal 82 displays an object 950 for viewing related information based on the related information received from the server 40 according to a control of application software.

Each object 910 of objects 910a to 910e includes information indicating a menu name and information indicating a priority. For example, the object 910a includes information 912 on a menu name and information 916 on a priority.

The object 910d indicates the recommended meal menu associated with the exercise information after having this meal. The object 910d includes the object 930 indicating the exercise content recommended when the user has this recommended meal menu.

The object 910e indicates a recommended meal menu associated with a recommended meal menu for a next meal. The object 910e includes the object 940 indicating a meal menu recommend to a next meal after having a meal of this recommended meal menu.

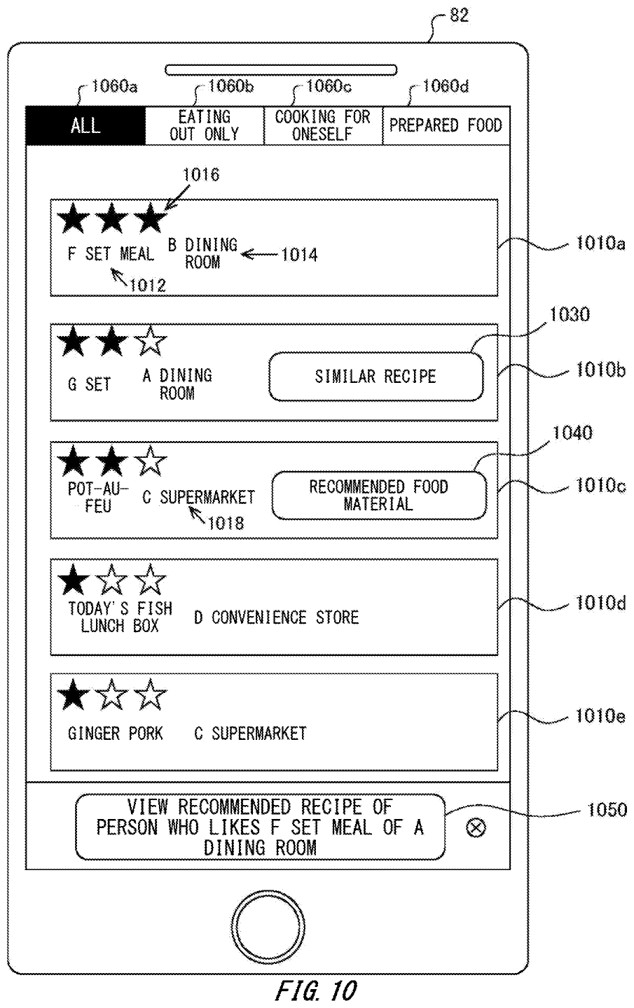

FIG. 10 schematically illustrates display contents of a recommended meal menu displayed on a user terminal 82. FIG. 10 illustrates one example of a display screen of a recommended meal menu for dinner when a current position of the user 80 is not in a particular restaurant.

The user terminal 82 displays objects 1010a to 1010e indicating recommended meal menus based on information indicating a recommended meal menu received from the server 40 according to a control of application software. The user terminal 82 displays each of the objects 1010a to 1010e in an order of priority. The user terminal 82 displays the object 1050 for viewing related information based on the related information received from the server 40 according to a control of application software.

The object 1010a and the object 1010b indicate recommended meal menus when the user has a meal outside a home. The object 1010c, the object 1010d, and the object 1010e indicate recommended meal menus when a user has the meal at home. The object 1010c and the object 1010e indicate recommended meal menus including foods obtained by cooking at home. The object 1010d indicates a recommended meal menu including a premade food.

Each object 1010 of the objects 1010a to 1010e includes information indicating a menu name and information indicating a priority. For example, the object 1010a includes information 1012 on a menu name, information 1014 on a restaurant name, and information 1016 on a priority. The objects 1010c to 1010d include information indicating a name of a store where the user can purchase a food material or a food. For example, the object 1010c includes information 1018 on a name of a store where the user can purchase a food material.

The object 1010b includes the object 1030 for referring to a recipe for self-cooking a meal of a similar meal menu to this recommended meal menu. The object 1010c includes the object 1040 for referring to a recommended food material for preparing this recommended meal menu.

The objects 1060a to 1060d are objects relating to the setting of a narrow-down condition of displayed recommended meal menus. The object 1060a cancels the narrow-down condition. The object 1060b causes the user terminal to display only recommended meal menus on the screen when the user has a meal outside a home. The object 1060c causes the user terminal to display only recommended meal menus including a food obtained by cooking on the screen. The object 1060d causes the user terminal to display only recommended meal menus including a cooked food on the screen. When one of the objects 1060a to 1060d is selected, the user terminal 82 displays only a recommended meal menu that meets a condition associated with the selected object.

Using the system 10 described above, a meal menu suitable for the user 80 can be recommended based on a meal history of individual user 80.

In particular, it is possible to recommend a meal menu specialized for individual user 80 based on goals, taste, and preference of users 80.

The system 10 of the present embodiment includes the database 50. However, it is possible to adopt a form not including the database 50. That is, the server 40 can operate as a system having functions of acquiring, from an external database, information indicating past meal history of the user 80, determining a meal menu to be recommended to the user 80 from a plurality of pre-registered meal menus based on the acquired information, and providing information indicating the determined meal menu. The user terminal 82 may include at least some of functions of the server 40. That is, the user terminal 82 alone can operate as a system having functions of acquiring information indicating past meal history of the user 80, determining a meal menu to be recommended to the user 80 from a plurality of pre-registered meal menus based on the acquired information, and providing information indicating the determined meal menu.

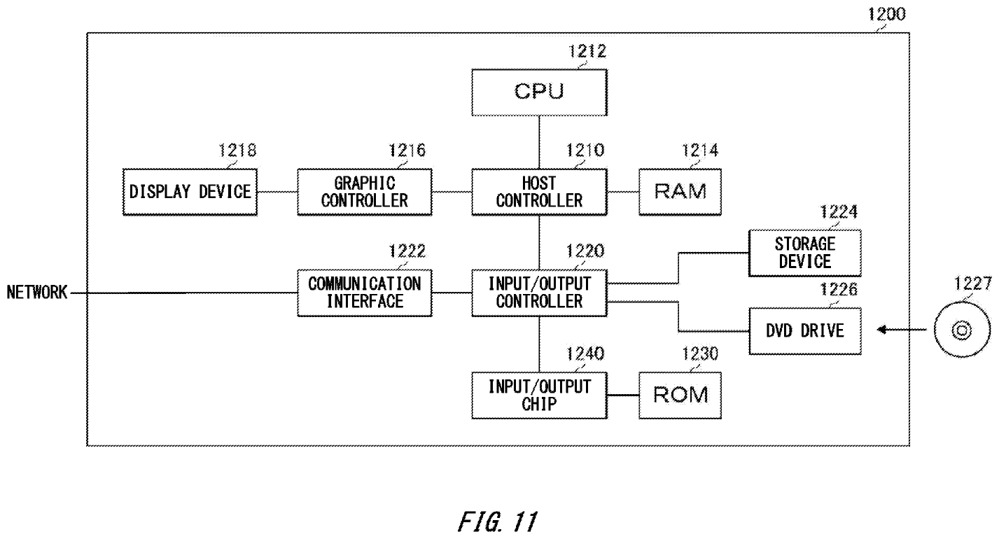

FIG. 11 schematically illustrates one example of a hardware configuration of a computer 1200 functioning as a server 40. A program installed on a computer 1200 can cause a computer 1200 to function as one or more “units” of a device according to the present embodiment, or cause the computer 1200 to execute an operation relating to the device according to the present embodiment or the one or more “units,” and/or cause the computer 1200 to execute a process according to the present embodiment or a stage of the process. Such program can be executed by a CPU 1212 so that the program causes the computer 1200 to execute specific operations associated with some or all of blocks in a flowchart and a block diagram described herein.

The computer 1200 of the present embodiment includes a CPU 1212, an RAM 1214, and a graphics controller 1216 and they are connected with one another via a host controller 1210. The computer 1200 includes an input/output unit such as a communication interface 1222, a storage device 1224, a DVD drive 1226, and an IC card drive and they are connected to the host controller 1210 via an input/output controller 1220. A DVD drive 1226 may be a DVD-ROM drive, a DVD-RAM drive and the like. The storage device 1224 may be a hard disk drive, a solid-state drive and the like. The computer 1200 also includes legacy input/output units such as a ROM 1230 and a keyboard and they are connected to the input/output controller 1220 through an input/output chip 1240.

The CPU 1212 operates according to programs stored in the ROM 1230 and the RAM 1214, thereby controlling each unit. The graphic controller 1216 obtains image data generated by the CPU 1212 on a frame buffer or the like provided in the RAM 1214 or in itself, and causes the image data to be displayed on the display device 1218.

The communication interface 1222 communicates with other electronic devices via a network. The storage device 1224 stores a program and data used by a CPU 1212 in the computer 1200. The DVD drive 1226 reads a program and data from the DVD-ROM 1227 or the like and provides the read program or data to the storage device 1224. The IC card drive reads programs and data from the IC card, and/or writes programs and data into the IC card.

The ROM 1230 stores therein a boot program or the like executed by the computer 2200 at the time of activation, and/or a program depending on a hardware of the computer 1200. The input/output chip 1240 allows various input/output units to be connected to an input/output controller 1220 via a USB port, a parallel port, a serial port, a keyboard port, a mouse port and the like.

The program is in a computer-readable storage medium such as a DVD-ROM 1227 or an IC card. The program is read from the computer-readable storage medium, installed on a storage device 1224, an RAM 1214, or a ROM 1230 that is also an example of the computer-readable storage medium, and is executed by the CPU 1212. The information processing described in these programs is read into the computer 2200, resulting in cooperation between a program and the above-mentioned various types of hardware resources. Devices or methods may be constituted by implementing the operations or processing on information through the use of the computer 1200.

For example, when communication is performed between the computer 1200 and an external device, the CPU 1212 may execute a communication program loaded onto the RAM 1214 to instruct communication processing to the communication interface 1222, based on the processing described in the communication program. Under a control of the CPU 1212, the communication interface 1222 reads transmission data stored in a transmission buffer area provided on a recording medium such as an RAM 1214, a storage device 1224, a DVD-ROM 1227, or an IC card, transmits the read transmission data to the network, or writes data received from the network to a reception buffer area or the like provided on the recording medium.

The CPU 1212 may cause the RAM 1214 to read all or necessary portions of a file or a database stored in an external recording medium such as a storage device 1224, a DVD drive 1226 (DVD-ROM 1227), and an IC card and execute various types of processes to data on the RAM 1214. The CPU 1212 may then write back the processed data to the external recording medium.

Various types of pieces of information, such as various types of programs, data, tables, and databases, may be stored in the recording medium to undergo information processing. The CPU 1212 may execute various types of processes on the data read from the RAM 1214, which include various types of operations, processing of information, condition judging, conditional branch, unconditional branch, search/replace of information, etc., as described throughout this disclosure and designated by an instruction sequence of programs, and writes the result back to the RAM 1214. In addition, the CPU 1212 may search for information in a file, a database, and the like in the recording medium. For example, when each of a plurality of entries having an attribute value of a first attribute associated with an attribute value of a second attribute is stored in a recording medium, from the plurality of entries, the CPU 1212 may search for an entry matching with a condition in which an attribute value of a first attribute is specified, read an attribute value of a second attribute stored in the entry, thereby acquiring an attribute value of a second attribute associated with the first attribute satisfying the predetermined condition.

The above described program or a software module may be stored on a computer 1200 or a computer-readable storage medium near the computer 1200. In addition, a recording medium such as a hard disk or a RAM provided in a server system connected to a dedicated communication network or the Internet can be used as the computer readable media, thereby providing the program to the computer 1200 via the network.

Blocks in the flowchart and the block diagram of the present embodiment may represent a stage of a process of an operation or a “unit” of a device responsible for performing an operation. Certain stages and “units” may be implemented by dedicated circuit, programmable circuit supplied with computer-readable instructions stored on computer-readable storage media, and/or processors supplied with computer-readable instructions stored on computer-readable storage media. The dedicated circuit may include a digital and/or analog hardware circuit and may include an integrated circuit (IC) and/or a discrete circuit. Programmable circuit may include reconfigurable hardware circuits comprising logical AND, OR, XOR, NAND, NOR, and other logical operations, flip-flops, registers, memory elements, etc., such as field-programmable gate arrays (FPGA), programmable logic arrays (PLA), etc., for example.

Computer-readable media may include any tangible device that can store instructions for execution by a suitable device, such that the computer-readable medium having instructions stored therein comprises an article of manufacture including instructions which can be executed to create means for performing operations specified in the flowcharts or block diagrams. Examples of computer-readable media may include an electronic storage medium, a magnetic storage medium, an optical storage medium, an electromagnetic storage medium, a semiconductor storage medium and the like. More specific examples of computer-readable media may include a floppy disk (registered trademark), a diskette, a hard disk, a random access memory (RAM), a read-only memory (ROM), an erasable programmable read-only memory (EPROM or Flash memory), an electrically erasable programmable read-only memory (EEPROM), a static random access memory (SRAM), a compact disc read-only memory (CD-ROM), a digital versatile disk (DVD), a BLU-RAY (registered trademark) disc, a memory stick, an integrated circuit card and the like.

Computer-readable instructions may include assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, JAVA (registered trademark), C++ and conventional procedural programming languages, such as the “C” programming language or similar programming languages.

The computer-readable instruction may be provided to a processor of a general purpose computer, a special purpose computer, other programmable data processing devices, or a programmable circuit locally or via a Wide Area Network (WAN), such as a Local Area Network (LAN) and the Internet so that a processor of a general purpose computer, a special purpose computer, other programmable data processing devices, or a programmable circuit can execute the computer-readable instruction to generate means for performing operation specified in a flowchart or a block diagram. Examples of processors include computer processors, processing units, microprocessors, digital signal processors, controllers, microcontrollers and the like.

While the embodiments of the present invention have been described above, the technical scope of the present invention is not limited to a scope described in the above described embodiments. It is apparent to persons skilled in the art that various alterations and improvements can be added to the above-described embodiments. It is also apparent from the scope of the claims that the embodiments added with such alterations or improvements can be included in the technical scope of the present invention.

The operations, procedures, steps, and stages of each process performed by an apparatus, system, program, and method shown in the claims, the specification, or diagrams can be performed in any order as long as the order is not indicated by “prior to,” “before,” or the like and as long as the output from a previous process is not used in a later process. Even if the process flow is described using phrases such as “first” or “next” in the claims, the specification, or diagrams, it does not necessarily mean that the process must be performed in this order.

### EXPLANATION OF REFERENCES

10: system, 20: network: 40: server: 42: processing device: 46: communication device: 48: storage device: 50: database: 80: user, 82: user terminal, 210: information acquiring unit, 220: identification unit, 230: determination unit, 240: provision unit, 250: storage control unit, 260: image acquiring unit, 910: object, 912: information, 916: information, 930: object, 940: object, 950: object, 1010: object, 1012: information, 1014: information, 1016: information, 1018: information, 1030: object, 1040: object, 1050: object, 1060: object, 1200: computer, 1210: host controller, 1212: CPU, 1214: RAM, 1216: graphics controller, 1218: display device, 1220: input/output controller, 1222: communication interface, 1224: storage device, 1226: DVD drive, 1227: DVD-ROM, 1230: ROM, 1240: input/output chip

What is claimed is:

1. A system comprising:
an information acquiring unit that acquires information indicating a past meal history of a user;
a determination unit that determines a meal menu recommend to the user from a plurality of pre-registered meal menus based on the information acquired by the information acquiring unit; and
a provision unit that provides information indicating a meal menu determined by the determination unit.

2. The system according to claim 1, wherein
the information acquiring unit further acquires information indicating a goal of the user; and
the determination unit determines a meal menu that is suited to the goal of the user from the plurality of meal menus based further on the information indicating the goal of the user.

3. The system according to claim 1, the system further comprising:
an image acquiring unit that acquires information indicating an image of a food had by the user;
an identification unit that identifies a nutrient taken from the food by recognizing the food taken by the user from the image; and
a storage control unit that stores, as information indicating the meal history, and in a storage unit, information including nutritional information indicating a nutrient taken by the user, identification information of the user, and date and time information when the user has the food in association with one another, wherein
as the information indicating the meal history, the information acquiring unit acquires the nutritional information and the date and time information stored in the storage unit in association with the identification information of the user.

4. The system according to claim 1, wherein
the provision unit determines a meal menu recommend to the user from the plurality of menus that a store is ready to serve when the user is in a store providing a meal; and
determines a meal menu recommend to the user from a plurality of menus that can be served in a plurality of stores associated with a current position of the user when the user is not in a store providing a meal.

5. The system according to claim 1, wherein
the determination unit determines a first meal menu recommend when the user has a meal at a living location of the user and a second meal menu recommended when the user has a meal outside the living location.

6. The system according to claim 5, wherein
the provision unit provides, to the user, the first meal menu and the second meal menu according to an order of priority based on a current position of the user.

7. The system according to claim 6, wherein
the provision unit provides, to the user, the first meal menu and the second meal menu according to the order of priority based on a distance between the current position and the living location.

8. The system according to claim 6, wherein
as a distance between the current position and the living location increases, the provision unit sets a higher priority given to provision of the second meal menu to the user than a priority given to provision of the first meal menu.

9. The system according to claim 5, wherein
the provision unit provides, to the user, the first meal menu and the second meal menu according to an order of priority based on a current position of the user and a current time.

10. The system according to claim 9, wherein
the provision unit provides, to the user, the first meal menu and the second meal menu according to an order of priority based on a distance between the current position and the living location and the current time.

11. The system according to claim 9, wherein
the meal history includes information indicating a time when the user has a past meal; and
the provision unit sets a higher priority given to provision of the second meal menu to the user than a priority given to provision of the first meal menu according to an order of priority determined based on a time required for moving from the current position to the living location, a current time and a time when the user has a past meal.

12. The system according to claim 5, wherein
the first meal menu includes a third meal menu including a food obtained by cooking a food material and a fourth meal menu using a premade food.

13. The system according to claim 12, wherein
the provision unit provides, to the user, the third meal menu and the fourth meal menu according to an order of priority determined based on a distance between a current position and the living location of the user and the current time.

14. The system according to claim 1, wherein
the determination unit determines a meal menu recommend to the user from the plurality of meal menus based on information acquired by the information acquiring unit and information indicating a behavioral plan about a meal of the user; and
the provision unit provides, to the user, information indicating a meal menu determined by the determination unit, the information indicating the meal menu being provided in association with the information indicating the behavioral plan.

15. The system according to claim 14, wherein
the information indicating the behavioral plan includes information indicating a meal menu to take after the user has a next meal.

16. The system according to claim 14, wherein
the information indicating the behavioral plan includes information indicating an amount of exercise performed by the user after taking a next meal.

17. The system according to claim 1, wherein
the information acquiring unit further acquires information indicating a past exercise history of the user; and
the determination unit determines a meal menu recommend to the user from the plurality of meal menus based further on information indicating the past exercise history of the user.

18. The system according to claim 1, wherein
the information acquiring unit further acquires information indicating a past meal history of one or more other users associated with the user; and
the determination unit determines the recommended meal menu to the user among the plurality of meal menus based further on information indicating a past meal history of the one or more other users.

19. A non-transitory computer readable storage medium storing a program that causes a computer to function as:
an information acquiring unit that acquires information indicating a past meal history of a user;
a determination unit that determines a recommend meal menu to the user among a plurality of meal menus pre-registered based on information acquired by the information acquiring unit; and
a provision unit that provides information indicating a meal menu determined by the determination unit.

20. A method performed by a computer, the method comprising:
acquiring information indicating a past meal history of a user:
determining a meal menu recommended to the user among a plurality of meal menus pre-registered based on the information acquired; and
providing information indicating the meal menu determined.
